(19) United States
(12) Patent Application Publication
(10) Pub. No.: US 2024/0428591 A1
(43) Pub. Date: Dec. 26, 2024

(54) SYSTEM AND METHOD FOR SURVEILLING A SCAN ROOM OF AN IMAGING SYSTEM FOR ABNORMAL ENVIRONMENTAL FACTORS

(71) Applicant: GE Precision Healthcare LLC, Waukesha, WI (US)

(72) Inventor: Chelsey Amanda Lewis, Waukesha, WI (US)

(21) Appl. No.: 18/337,585

(22) Filed: Jun. 20, 2023

(51) Int. Cl. G06V 20/52; A61B 6/03; G06V 20/40; G16H 40/60

(52) U.S. Cl. CPC G06V 20/52; A61B 6/032; G06V 20/44; G16H 40/60

(57) ABSTRACT

A computer-implemented method for monitoring an environment within a scan room having a medical imaging scanner includes receiving, at a processor, surveillance image data of the scan room from an optical imaging system disposed within the scan room. The computer-implemented method also includes identifying, via the processor, any abnormal factor or event within the environment that potentially adversely impacts a scan of a subject with the medical imaging scanner based on the surveillance image data. The computer-implemented method further includes causing, via the processor, a notification of an identified abnormal factor or event to be provided to an operator of the medical imaging scanner. The computer-implemented method even further includes recording, via the processor, any identified abnormal factor or event to an event log file.

### BACKGROUND

The subject matter disclosed herein relates to medical imaging systems and, more particularly, to a system and a method for surveilling a scan room of an imaging system for abnormal environmental factors.

In computed tomography (CT), X-ray radiation spans an object or a subject of interest being scanned, such as a human patient, baggage, or other object, and a portion of the radiation impacts a detector where the image data is collected. In digital X-ray systems a photodetector produces signals representative of the amount or intensity of radiation impacting discrete pixel regions of a detector surface. The signals may then be processed to generate an image that may be displayed for review. In the images produced by such systems, it may be possible to identify and examine the internal structures and organs within a subject's body. In CT imaging systems a detector array, including a series of detector elements or sensors, produces similar signals through various positions as a gantry is displaced around a subject or object being imaged, allowing volumetric image reconstructions to be obtained.

Currently there is little done for monitoring the environment surrounding a CT scanner in a scan room. Typically, a technician visually inspects the scan room for abnormalities (e.g., through a lead window, on a closed-circuit television system, or by entering the scan room). These abnormalities may include a dirty scan window, fluids from patients or contrast media spilled on a scan window, unwanted items (e.g., chair, stretcher, etc.) in the scan room or adjacent a table of the CT scanner, potential contamination, and/or whether everything appears clean. An abnormality in the scan room may affect image quality, patient safety, and/or cause an undesired incident during the imaging procedure. In addition, some of these potential abnormalities may not be detected. Further, these potential abnormalities may not be catalogued for later reference.

### SUMMARY

Certain embodiments commensurate in scope with the originally claimed subject matter are summarized below. These embodiments are not intended to limit the scope of the claimed subject matter, but rather these embodiments are intended only to provide a brief summary of possible forms of the subject matter. Indeed, the subject matter may encompass a variety of forms that may be similar to or different from the embodiments set forth below.

In one embodiment, a computer-implemented method for monitoring an environment within a scan room having a medical imaging scanner is provided. The computer-implemented method includes receiving, at a processor, surveillance image data of the scan room from an optical imaging system disposed within the scan room. The computer-implemented method also includes identifying, via the processor, any abnormal factor or event within the environment that potentially adversely impacts a scan of a subject with the medical imaging scanner based on the surveillance image data. The computer-implemented method further includes causing, via the processor, a notification of an identified abnormal factor or event to be provided to an operator of the medical imaging scanner. The computer-implemented method even further includes recording, via the processor, any identified abnormal factor or event to an event log file.

In another embodiment, a system for monitoring for monitoring an environment within a scan room having a computed tomography scanner is provided. The system includes a memory encoding processor-executable routines. The system also includes a processor configured to access the memory and to execute the processor-executable routines, wherein the processor-executable routines, when executed by the processor, cause the processor to perform actions. The actions include receiving surveillance image data of the scan room from the optical imaging system. The actions also include identify any abnormal factor or event within the environment that potentially adversely impacts a scan of a subject with the computed tomography scanner based on the image surveillance data. The actions further include recording any identified abnormal factor or event to an event log file.

In a further embodiment, a non-transitory computer-readable medium is provided. The computer-readable medium includes processor-executable code that when executed by a processor, causes the processor to perform actions. The actions include receiving surveillance image data of a scan room having a medical imaging scanner from an optical imaging system disposed within the scan room. The actions also include identifying any abnormal factor or event within an environment of the scan room that potentially adversely impacts a scan of a subject with the medical imaging scanner based on the surveillance image data. The actions further include causing a notification of an identified abnormal factor or event to be provided to an operator of the medical imaging scanner. The actions even further include recording any identified abnormal factor or event to an event log file.

### BRIEF DESCRIPTION OF THE DRAWINGS

These and other features, aspects, and advantages of the present disclosed subject matter will become better understood when the following detailed description is read with reference to the accompanying drawings in which like characters represent like parts throughout the drawings, wherein:

FIG. 1 is a pictorial representation of a CT imaging system, in accordance with aspects of the present disclosure;

FIG. 2 is a block diagram of the CT imaging system in FIG. 1, in accordance with aspects of the present disclosure;

FIG. 3 is a schematic diagram of a scan room having a medical imaging scanner and optical imaging system (e.g., camera on scanner), in accordance with aspects of the present disclosure;

FIG. 4 is a schematic diagram of a scan room having a medical imaging scanner and optical imaging system (e.g., camera separate from scanner), in accordance with aspects of the present disclosure;

FIG. 5 is a schematic diagram of a scan room having a medical imaging scanner and optical imaging system (e.g., camera on scanner and camera separate from scanner), in accordance with aspects of the present disclosure;

FIG. 6 is a flowchart of a method for monitoring an environment within a scan room having a medical imaging scanner, in accordance with aspects of the present disclosure;

FIG. 7 is a flowchart of a method for identifying an abnormal factor or event in an environment within a scan room having a medical imaging scanner that potentially adversely impacts a scan (e.g., utilizing reference image data), in accordance with aspects of the present disclosure;

FIG. 8 is a flowchart of a method for identifying an abnormal factor or event in an environment within a scan room having a medical imaging scanner that potentially adversely impacts a scan (e.g., utilizing probability calculations), in accordance with aspects of the present disclosure;

FIG. 9 is a flowchart of a method for identifying an abnormal factor or event in an environment within a scan room having a medical imaging scanner that potentially adversely impacts a scan (e.g., utilizing both reference image data and probability calculations), in accordance with aspects of the present disclosure;

FIG. 10 is a flowchart of a method for monitoring an environment within a scan room having a medical imaging scanner (e.g., retroactively), in accordance with aspects of the present disclosure;

FIG. 11 is a schematic diagram of a user interface providing a notification of an abnormal factor or event identified in an environment within a scan room having a medical imaging scanner that potentially adversely impacts a scan, in accordance with aspects of the present disclosure; and

FIG. 12 is a schematic diagram of a user interface providing a corrective action for an abnormal factor or event identified in an environment within a scan room having a medical imaging scanner that potentially adversely impacts a scan, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While aspects of the following discussion may be provided in the context of medical imaging, it should be appreciated that the present techniques are not limited to such medical contexts. Indeed, the provision of examples and explanations in such a medical context is only to facilitate explanation by providing instances of real-world implementations and applications. However, the present approaches may also be utilized in other contexts, such as tomographic image reconstruction for industrial Computed Tomography (CT) used in non-destructive inspection of manufactured parts or goods (i.e., quality control or quality review applications), and/or the non-invasive inspection of packages, boxes, luggage, and so forth (i.e., security or screening applications). In general, the present approaches may be useful in any imaging or screening context to monitor for adverse environmental factors that affected or may affect a scan.

The present disclosure provides systems and methods for monitoring an environment within a scan room having a medical imaging scanner for any abnormal factor or event that may potentially adversely impact a scan of an object with the medical imaging scanner. In particular, the disclosed techniques include receiving surveillance image data of the scan room from an optical imaging system disposed within the scan room, identifying any abnormal factor or event within the environment that potentially adversely impacts a scan of an object with the medical imaging scanner based on the surveillance image data, and recording (e.g., logging) any identified abnormal factor or event to an event log file.

A number of different types of events may serve as an abnormal factor or event. For example, any variety of objects may be in the way when moving components (e.g., table) of the medical imaging scanner or located in an undesirable location (e.g., behind a gantry). For example, a boom, a chair, or straps may be in the way or impede the movement of a component of the medical imaging scanner (e.g., table or cradle). Another example of an abnormal factor or event may be a risk of pulling an object or device (e.g., ventilator tube, intravenous line, etc.) from the subject (e.g., patient) when moving the table. A further example of an abnormal event or factor would be an individual entering the scan room during a scan. An even further example of an abnormal event or factor may be contrast or bodily fluids located on the scan window. A yet further example of an abnormal event or factor may be a dent in a component of the medical imaging scanner. An abnormal factor or event potentially adversely impacts the scan by adversely impacting an image quality of an image data obtained during the scan, potentially adversely impacting a safety of the subject, and/or potentially adversely impacting setting up or conducting the scan.

The monitoring for adverse factors or events may occur prior to (e.g., during preparation and setup of scan) and/or during the scan. The monitoring may occur in the presence or the absence of the subject to be imaged. In certain embodiments, the identification of any abnormal factor or event may occur prior to the scan. In certain embodiments, the identification of any abnormal factor or event may occur during the scan. In certain embodiments, the identification of any abnormal factor or event may occur subsequent to the scan (e.g., retroactively). In certain embodiments, the identification of any abnormal factor event may occur by comparing current surveillance image data to past surveillance image data (e.g., serving as reference data). In certain embodiments, identification of any abnormal factor or event may occur by determining or calculating a probability that a particular factor or event may adversely affect the scan. In certain embodiments, if the probability meets a specific threshold, the particular factor or event may be classified as an abnormal factor or event.

In certain embodiments, the disclosed techniques include causing a notification of an identified abnormal factor or event to be provided to an operator of the medical imaging scanner. For example, a notification may be provided on an operator or workstation console (e.g., on a display) for the medical imaging system or another device. In certain embodiments, a calculated probability score for the abnormal factor or event may be provided along with the notification. In certain embodiments, the disclosed techniques include causing (e.g., via control signals) a corrective action for the operator to perform to rectify the identified abnormal factor or event to be provided to the operator of the medical imaging scanner. For example, the corrective action may be provided on the operator or workstation console (e.g., on a display) for the medical imaging system or another device. As an example, the operator may be instructed to clean contrast off the scan window or to move a chair. In certain embodiments, the disclosed techniques include proactively ceasing an action related to the scan before the identified abnormal factor or event adversely affects the scan. For example, the scan may be stopped if somebody enters the scan room. In another example, automatic movement of the table may be ceased and the operator may have to enter the scan room to manually move the table via the controls associated with the table.

The optical imaging system may include one or more cameras or sensors (e.g., video cameras, three-dimensional (3D) camera, light detection and ranging or laser imaging, detection, and ranging (LiDAR) camera, etc.) located within the scan room. In certain embodiments, one or more cameras may be disposed on a component of the medical imaging scanner. For example, a camera may be located on a top of a gantry. In certain embodiments, one or more cameras may be located within the scan room at a location separate from the medical imaging scanner. For example, a camera may be located on a ceiling in the scan room. In certain embodiments, cameras may be disposed on a component of the medical imaging scanner and at a location separate from the medical image scanner.

Although the following discusses the disclosed embodiments with regard to computed tomography (CT) imaging systems, the techniques described herein may apply to other types of imaging systems. For example, the disclosed techniques may apply to a magnetic resonance imaging (MRI) system or a nuclear medicine imaging system such as a positron emission tomography (PET) imaging system or a single photon emission computed tomography (SPECT) imaging system. The disclosed techniques may also apply to medical imaging systems having a combination of the above medical imaging modalities.

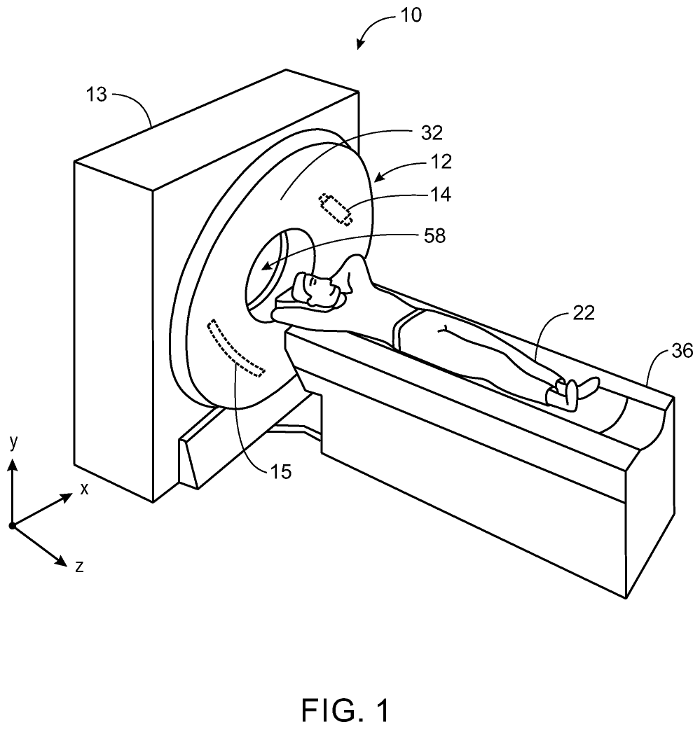

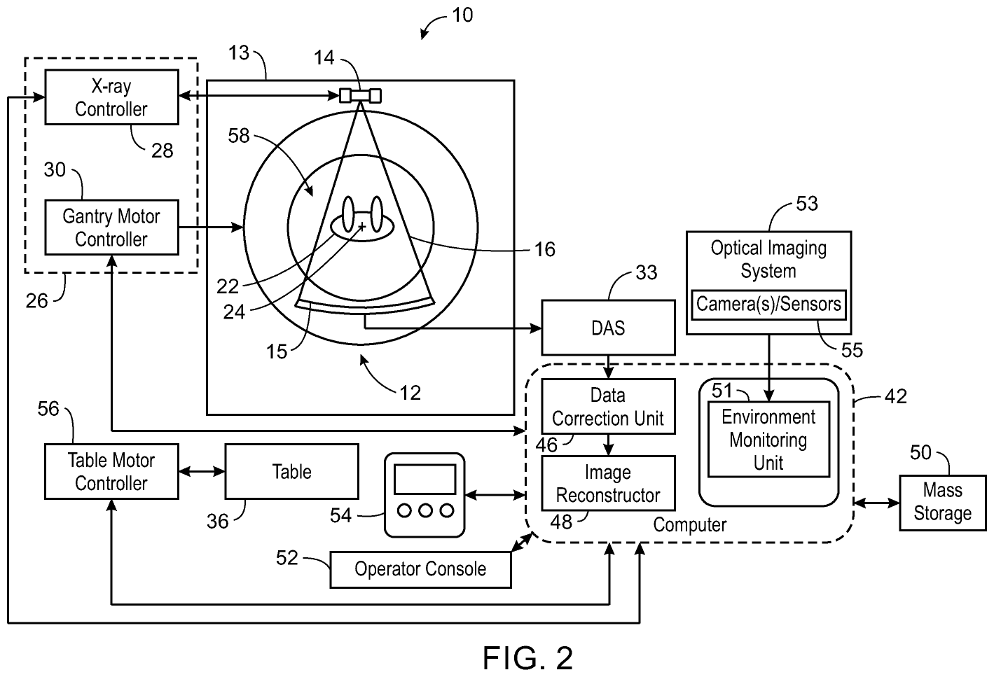

With the preceding in mind and referring to FIGS. 1 and 2, a CT imaging system 10 is shown, by way of example. The CT imaging system 10 of FIGS. 1 and 2 may be utilized with a dual-modality imaging system (e.g., positron emission tomography (PET)/CT imaging system). The CT imaging system 10 (e.g., CT scanner) includes a gantry 12 coupled having a housing 13 (e.g., gantry housing). The gantry 12 has a rotating component and a stationary component. The gantry 12 has an X-ray source 14 that projects a beam of X-rays 16 toward an X-ray detector assembly or X-ray detector array 15 (e.g., having a plurality of detector modules) on the opposite side of the gantry 12. The X-ray source 14 and the X-ray detector assembly 15 are disposed on the rotating portion of the gantry 12. The X-ray detector assembly 15 is coupled to data acquisition systems (DAS) 33. The plurality of detector modules of the X-ray detector assembly 15 detect the projected X-rays that pass through a patient or subject 22, and DAS 33 converts the data to digital signals for subsequent processing. Each detector module of the X-ray detector assembly 15 in a conventional system produces an analog electrical signal that represents the intensity of an incident X-ray beam and hence the attenuated beam as it passes through the patient 22. During a scan to acquire X-ray projection data, gantry 12 and the components mounted thereon rotate about a center of rotation 24 (e.g., isocenter) so as to collect attenuation data from a multitude of view angles relative to the imaged volume.

Rotation of gantry 12 and the operation of X-ray source 14 are governed by a control mechanism 26 of CT system 10. Control mechanism 26 includes an X-ray controller 28 that provides power and timing signals to the X-ray source 14 and a gantry motor controller 30 that controls the rotational speed and position of gantry 12.

A computer 42 (separate from or a part of the CT imaging system 10) includes a data correction unit 46 for processing or correcting the CT scan data from the DAS 33. The computer 42 also includes an image reconstructor 48. The image reconstructor 48 receives sampled and digitized X-ray data from DAS 33 and performs high-speed reconstruction. The reconstructed image is applied as an input to the computer 42, which stores the image in a mass storage device 50. The computer 42 further includes an environment monitoring unit 41. The environment monitoring unit 51 is configured to receive surveillance image data of a scan room having the CT imaging system 10 from an optical imaging system 53 disposed within the scan room and to monitor for any abnormal factor or event within the environment of the scan room that may potentially adversely impact a scan of the patient 22.

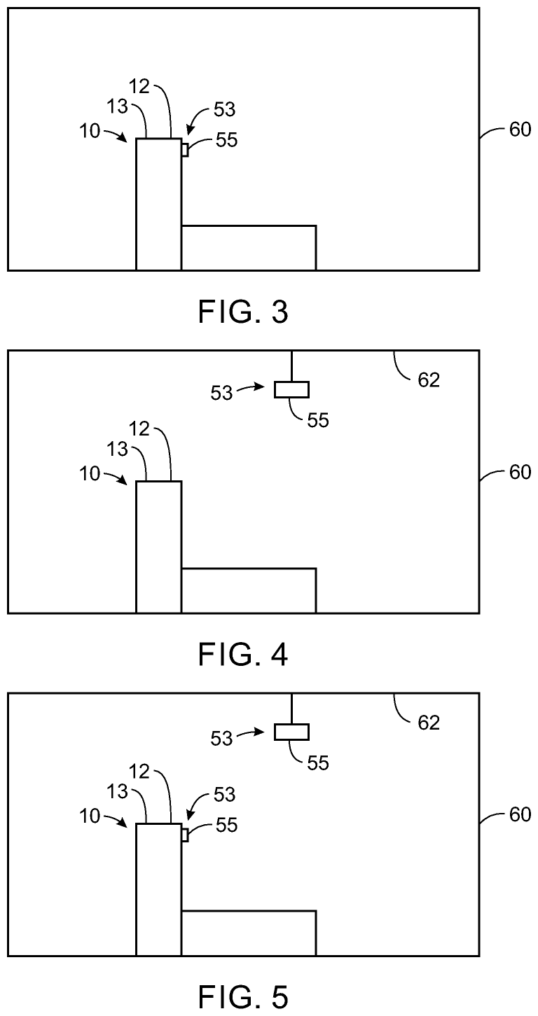

The optical imaging system 53 may include one or more cameras or sensors 55 (e.g., video cameras, three-dimensional (3D) camera, LiDAR camera, etc.) located within the scan room. FIGS. 3-5 are schematic diagrams of a scan room 60 having a medical imaging scanner within (e.g., CT imaging system 10). In certain embodiments, one or more cameras 55 may be disposed on a component of the medical imaging scanner. As depicted in FIG. 3, a single camera 55 is disposed at a top of the housing 13 of the gantry 12 of the CT imaging system 10. More than one camera 55 may be disposed on components of the CT imaging system 10. As depicted in FIG. 4, a single camera 55 is located within the scan room 60 at a location separate from the CT imaging system. In particular, a single camera 55 is located on a ceiling 62 of the scan room 60. In certain embodiments, the camera 55 may be located on a different wall of the scan room 60 or another structure in the scan room 60 that is separate from the CT imaging system 10. In certain embodiments, more than one camera 55 may be located within the scan room 60 at different locations separate from the CT imaging system 10. As depicted in FIG. 5, cameras 55 may be disposed on a component of the CT imaging system 10 (e.g., top of the housing 13 of the gantry 12) and at a location separate from the CT imaging system 10 (e.g., the ceiling 62).

Returning to FIGS. 1 and 2, computer 42 also receives commands and scanning parameters from an operator via console 52. An associated display 54 allows the operator to observe the reconstructed image and other data from the computer 42. For example, the associated display 54 may display a notification of an identified abnormal factor or event in the scan room that may potentially adversely impacts the scan of the patient 22. The notification may identify the abnormal factor or event. The notification may also provide a calculated probability (e.g., probability score) that a particular factor or event would adversely impact the scan. In certain embodiments, the associated display 54 may display a corrective action for the operator to perform to rectify the identified abnormal factor or event to be provided to the operator of the CT imaging system 10. In certain embodiments, the associated display 54 may display an action automatically taken (e.g., action ceased) proactively by the monitoring unit 51 to cease an action related to the scan (e.g., cease the scan, cease movement of the table 36). The operator supplied commands and parameters are used by computer 42 to provide control signals and information to the DAS 33, X-ray controller 28, and gantry motor controller 30. In addition, computer 42 operates a table motor controller 56, which controls a motorized table 36 to position the patient 22 relative to the gantry 12. Particularly, table 36 moves portions of the patient 22 through a gantry opening or bore 58.

The computer 42 include includes processing circuitry. The processing circuitry may be one or more general or application-specific microprocessors. The processing circuitry may be configured to execute instructions stored in a memory to perform various actions. For example, the processing circuitry may be utilized for receiving surveillance image data of the scan room from an optical imaging system disposed within the scan room, identifying any abnormal factor or event within the environment that potentially adversely impacts a scan of an object with the CT imaging system 10 based on the surveillance image data, and recording (e.g., logging) any identified abnormal factor or event to an event log file. The processing circuitry may also be utilized for monitoring for adverse factors or events prior to (e.g., during preparation and setup of scan) and/or during the scan. The monitoring may occur in the presence or the absence of the subject to be imaged. The processing circuitry may also be utilized for identifying any abnormal factor or event that may occur prior to the scan. The processing circuitry may also be utilized for identifying any abnormal factor or event that may occur during the scan. The processing circuitry may also be utilized for retroactively identifying any abnormal factor or event may occur subsequent to the scan. In certain embodiments, the processing circuitry may identify any abnormal factor event may occur by comparing current surveillance image data to past surveillance image data (e.g., serving as reference data). In certain embodiments, the processing circuitry may identify any abnormal factor or event may occur by determining or calculating a probability that a particular factor or event may adversely affect the scan. In certain embodiments, if the probability meets a specific threshold, the particular factor or event may be classified as an abnormal factor or event.

Further, the processing circuitry may be utilized for causing a notification of an identified abnormal factor or event to be provided to an operator of the CT imaging system 10. The processing circuitry may further be utilized for causing (e.g., via control signals) a corrective action for the operator to perform to rectify the identified abnormal factor or event to be provided to the operator of the CT imaging system 10. Still further, the processing circuitry may be utilized for proactively ceasing an action related to the scan before the identified abnormal factor or event adversely affects the scan. For example, the scan may be stopped if somebody enters the scan room. In another example, automatic movement of the table may be ceased and the operator may have to enter the scan room to manually move the table via the controls associated with the table.

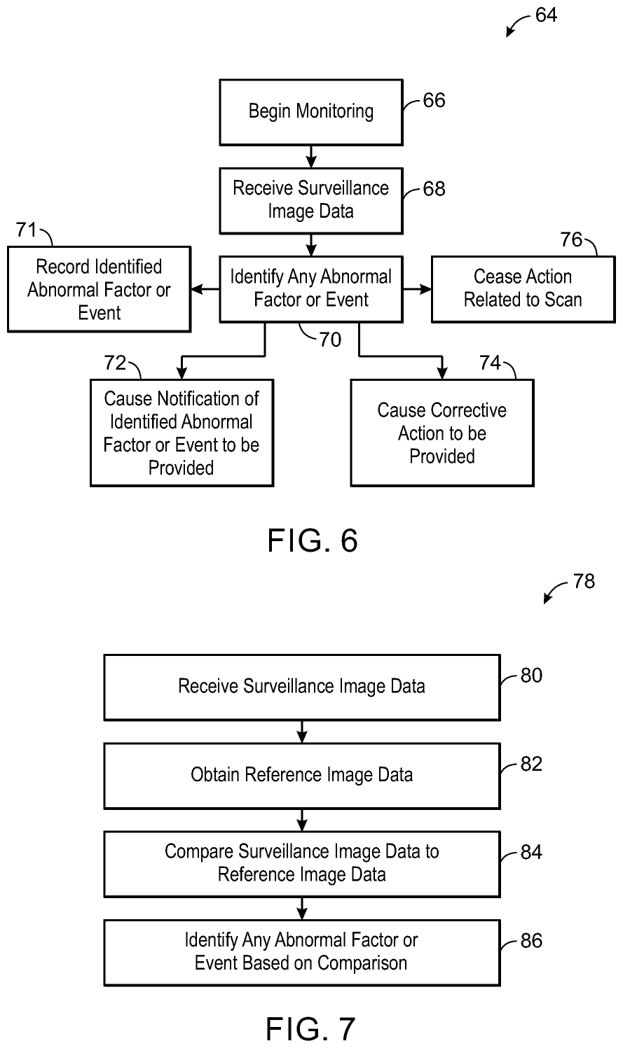

FIG. 6 is a flowchart of a method 64 for monitoring an environment within a scan room having a medical imaging scanner. The method 64 may be performed by one or more components (e.g., processing circuitry) of the CT imaging system 10 in FIGS. 1 and 2 or another type of imaging system. One or more steps of the method 64 may be performed simultaneously and/or in a different order than depicted in FIG. 6.

The method 64 includes beginning monitoring an environment within a scan room having a medical imaging scanner (block 66). The method 64 also includes receiving or obtaining surveillance image data of the scan room from an optical imaging system disposed within the scan room (block 68). The optical imaging system may include one or more cameras or sensors (e.g., video cameras, three-dimensional (3D) camera, LiDAR camera, etc.) located within the scan room. In certain embodiments, one or more cameras may be disposed on a component of the medical imaging scanner. For example, a camera may be located on a top of a gantry. In certain embodiments, one or more cameras may be located within the scan room at a location separate from the medical imaging scanner. For example, a camera may be located on a ceiling in the scan room. In certain embodiments, cameras may be disposed on a component of the medical imaging scanner and at a location separate from the medical image scanner. In certain embodiments, the surveillance image data is video data. In certain embodiments, the surveillance image data is individual frames or images.

The method 64 further includes identifying any abnormal factor or event within the environment of the scan room that potentially adversely impacts a scan of a subject (e.g., patient) with the medical imaging scanner based on the surveillance image data (block 70). In certain embodiments, a probability may be calculated for a particular event in order to identify a factor or event as an abnormal factor or event that potentially adversely impacts the scan. In certain embodiments, comparing current surveillance image data of the scan room to reference image data (e.g., where environment conditions are considered normal) may be utilized in identifying a factor or event as an abnormal factor or event that potentially adversely impacts the scan. An abnormal factor or event potentially adversely impacts the scan by adversely impacting an image quality of an image data obtained during the scan, potentially adversely impacting a safety of the subject, and/or potentially adversely impacting setting up or conducting the scan.

The monitoring for adverse factors or events may occur prior to (e.g., during preparation and setup of scan) and/or during the scan. The monitoring may occur in the presence or the absence of the subject to be imaged. In certain embodiments, the identification of any abnormal factor or event may occur prior to the scan. In certain embodiments, the identification of any abnormal factor or event may occur during the scan. In certain embodiments, the identification of any abnormal factor or event may occur subsequent to the scan (e.g., retroactively).

The method 64 even further includes recording any identified abnormal factor or event to an event log file (block 71). In certain embodiments, the event log files may be stored locally in a memory of the medical imaging system or the facility having the medical imaging system. In certain embodiments, the event log files may be stored in a memory or database remotely located form the medical imaging system or the facility having the medical imaging system (e.g., manufacturer or provider of the medical imaging system).

In certain embodiments, the method 64 includes causing a notification of an identified abnormal factor or event to be provided to an operator of the medical imaging scanner (block 72). For example, a notification may be provided on an operator or workstation console (e.g., on a display) for the medical imaging system or another device. In certain embodiments, a calculated probability score for the abnormal factor or event may be provided along with the notification.

In certain embodiments, the method 64 includes causing (e.g., via control signals) a corrective action for the operator to perform to rectify the identified abnormal factor or event to be provided to the operator of the medical imaging scanner (block 74). For example, the corrective action may be provided on the operator or workstation console (e.g., on a display) for the medical imaging system or another device. As an example, the operator may be instructed to clean contrast off the scan window or to move a chair. In certain embodiments, the method 64 includes proactively ceasing an action related to the scan before the identified abnormal factor or event adversely affects the scan (block 76). For example, the scan may be stopped if somebody enters the scan room. In another example, automatic movement of the table may be ceased and the operator may have to enter the scan room to manually move the table via the controls associated with the table.

FIG. 7 is a flowchart of a method 78 for identifying an abnormal factor or event in an environment within a scan room having a medical imaging scanner that potentially adversely impacts a scan (e.g., utilizing reference image data). The method 78 may be performed by one or more components (e.g., processing circuitry) of the CT imaging system 10 in FIGS. 1 and 2 or another type of imaging system. One or more steps of the method 78 may be performed simultaneously and/or in a different order than depicted in FIG. 7.

The method 78 includes receiving or obtaining surveillance image data of the scan room from an optical imaging system disposed within the scan room (block 80). The optical imaging system may include one or more cameras or sensors (e.g., video cameras, three-dimensional (3D) camera, LiDAR camera, etc.) located within the scan room. In certain embodiments, one or more cameras may be disposed on a component of the medical imaging scanner. For example, a camera may be located on a top of a gantry. In certain embodiments, one or more cameras may be located within the scan room at a location separate from the medical imaging scanner. For example, a camera may be located on a ceiling in the scan room. In certain embodiments, cameras may be disposed on a component of the medical imaging scanner and at a location separate from the medical image scanner. In certain embodiments, the surveillance image data is video data. In certain embodiments, the surveillance image data is individual frames or images.

The method 78 also includes obtaining reference image data of the scan room (block 82). The reference image data is of the scan room when environmental conditions are considered normal within the scan room. In certain embodiments, the reference image data is derived from an earlier time point prior to the scan. In certain embodiments, the reference image data is video data. In certain embodiments, the reference image data is individual frames or images.

The method 78 further includes comparing the surveillance image data to the reference image data (block 84). The method 78 even further identifying any abnormal factor or event within the environment of the scan room that potentially adversely impacts a scan of a subject (e.g., patient) with the medical imaging scanner based on the comparison (block 86). An abnormal factor or event potentially adversely impacts the scan by adversely impacting an image quality of an image data obtained during the scan, potentially adversely impacting a safety of the subject, and/or potentially adversely impacting setting up or conducting the scan. The monitoring for adverse factors or events may occur prior to (e.g., during preparation and setup of scan) and/or during the scan. The monitoring may occur in the presence or the absence of the subject to be imaged. In certain embodiments, the identification of any abnormal factor or event may occur prior to the scan. In certain embodiments, the identification of any abnormal factor or event may occur during the scan. In certain embodiments, the identification of any abnormal factor or event may occur subsequent to the scan (e.g., retroactively).

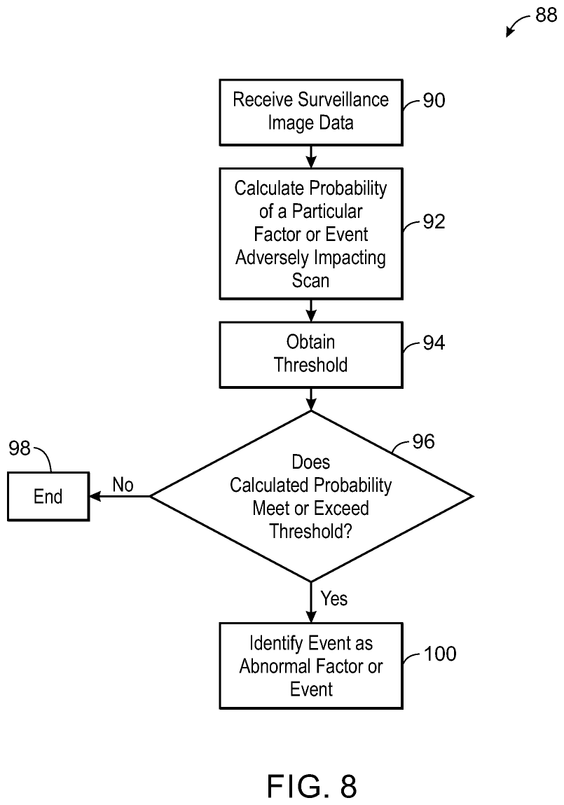

FIG. 8 is a flowchart of a method 88 for identifying an abnormal factor or event in an environment within a scan room having a medical imaging scanner that potentially adversely impacts a scan (e.g., utilizing probability calculations). The method 88 may be performed by one or more components (e.g., processing circuitry) of the CT imaging system 10 in FIGS. 1 and 2 or another type of imaging system. One or more steps of the method 88 may be performed simultaneously and/or in a different order than depicted in FIG. 8.

The method 88 includes receiving or obtaining surveillance image data of the scan room from an optical imaging system disposed within the scan room (block 90). The optical imaging system may include one or more cameras or sensors (e.g., video cameras, three-dimensional (3D) camera, LiDAR camera, etc.) located within the scan room. In certain embodiments, one or more cameras may be disposed on a component of the medical imaging scanner. For example, a camera may be located on a top of a gantry. In certain embodiments, one or more cameras may be located within the scan room at a location separate from the medical imaging scanner. For example, a camera may be located on a ceiling in the scan room. In certain embodiments, cameras may be disposed on a component of the medical imaging scanner and at a location separate from the medical image scanner. In certain embodiments, the surveillance image data is video data. In certain embodiments, the surveillance image data is individual frames or images.

The method 88 also includes calculating a probability (e.g., probability score) of a particular factor or event adversely impacting a scan with the medical imaging scanner in the scan room (block 92). The method 88 further includes obtaining a threshold or threshold value (block 94). The threshold or threshold value is a preset level where a factor or event is considered an abnormal factor or event that potentially aversely impacts the scan of the subject (e.g., patient) with the medical image scanner. The threshold or threshold value may be adjusted to a different preset level.

The method 88 yet further includes determining whether a calculated probability for a particular factor or event meets or exceeds the obtained threshold or threshold value (block 96). If the calculated probability for the particular factor or event does not at least meet the obtained threshold or threshold value, the method 88 includes ending the identification process with respect to the particular factor or event (without designating the particular factor or event abnormal) (block 98). If the calculated probability for the particular factor or event does meet or exceed the obtained threshold or threshold value, the method 88 includes identifying the particular factor or event as an abnormal factor or event that potentially adversely impacts the scan (block 100). An abnormal factor or event potentially adversely impacts the scan by adversely impacting an image quality of an image data obtained during the scan, potentially adversely impacting a safety of the subject, and/or potentially adversely impacting setting up or conducting the scan. The monitoring for adverse factors or events may occur prior to (e.g., during preparation and setup of scan) and/or during the scan. The monitoring may occur in the presence or the absence of the subject to be imaged. In certain embodiments, the identification of any abnormal factor or event may occur prior to the scan. In certain embodiments, the identification of any abnormal factor or event may occur during the scan. In certain embodiments, the identification of any abnormal factor or event may occur subsequent to the scan (e.g., retroactively).

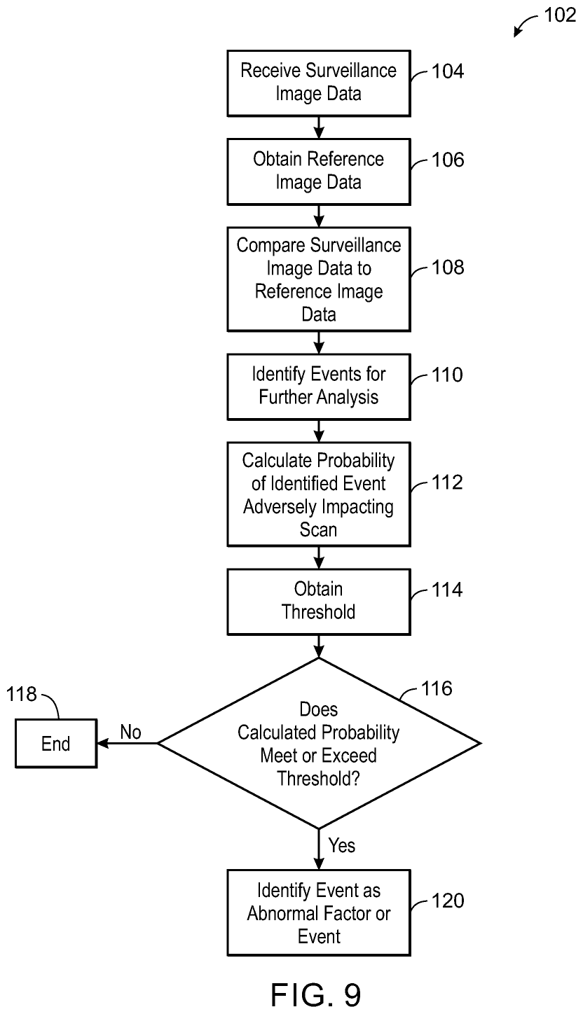

In certain embodiments, a combination of the methods 78 and 88 in the FIGS. 7 and 8, respectively, may be utilized. FIG. 9 is a flowchart of a method 102 for identifying an abnormal factor or event in an environment within a scan room having a medical imaging scanner that potentially adversely impacts a scan (e.g., utilizing both reference image data and probability calculations). The method 102 may be performed by one or more components (e.g., processing circuitry) of the CT imaging system 10 in FIGS. 1 and 2 or another type of imaging system. One or more steps of the method 102 may be performed simultaneously and/or in a different order than depicted in FIG. 9.

The method 102 includes receiving or obtaining surveillance image data of the scan room from an optical imaging system disposed within the scan room (block 104). The optical imaging system may include one or more cameras or sensors (e.g., video cameras, three-dimensional (3D) camera, LiDAR camera, etc.) located within the scan room. In certain embodiments, one or more cameras may be disposed on a component of the medical imaging scanner. For example, a camera may be located on a top of a gantry. In certain embodiments, one or more cameras may be located within the scan room at a location separate from the medical imaging scanner. For example, a camera may be located on a ceiling in the scan room. In certain embodiments, cameras may be disposed on a component of the medical imaging scanner and at a location separate from the medical image scanner. In certain embodiments, the surveillance image data is video data. In certain embodiments, the surveillance image data is individual frames or images.

The method 102 also includes obtaining reference image data of the scan room (block 106). The reference image data is of the scan room when environmental conditions are considered normal within the scan room. In certain embodiments, the reference image data is derived from an earlier time point prior to the scan. In certain embodiments, the reference image data is video data. In certain embodiments, the reference image data is individual frames or images.

The method 102 further includes comparing the surveillance image data to the reference image data (block 108). The method 102 even further identifying any factor or event within the environment of the scan room for further analysis based on the comparison (block 110).

The method 102 also includes calculating a probability (e.g., probability score) of the identified factor or event (from the comparison) adversely impacting a scan with the medical imaging scanner in the scan room (block 112). The method 102 further includes obtaining a threshold or threshold value (block 114). The threshold or threshold value is a preset level where a factor or event is considered an abnormal factor or event that potentially aversely impacts the scan of the subject (e.g., patient) with the medical image scanner. The threshold or threshold value may be adjusted to a different preset level.

The method 102 yet further includes determining whether a calculated probability for the identified factor or event meets or exceeds the obtained threshold or threshold value (block 116). If the calculated probability for the identified factor or event does not at least meet the obtained threshold or threshold value, the method 102 includes ending the identification process with respect to the particular factor or event (without designating the particular factor or event abnormal) (block 118). If the calculated probability for the identified factor or event does meet or exceed the obtained threshold or threshold value, the method 102 includes identifying the particular factor or event as an abnormal factor or event that potentially adversely impacts the scan (block 120). An abnormal factor or event potentially adversely impacts the scan by adversely impacting an image quality of an image data obtained during the scan, potentially adversely impacting a safety of the subject, and/or potentially adversely impacting setting up or conducting the scan. The monitoring for adverse factors or events may occur prior to (e.g., during preparation and setup of scan) and/or during the scan. The monitoring may occur in the presence or the absence of the subject to be imaged. In certain embodiments, the identification of any abnormal factor or event may occur prior to the scan. In certain embodiments, the identification of any abnormal factor or event may occur during the scan. In certain embodiments, the identification of any abnormal factor or event may occur subsequent to the scan (e.g., retroactively).

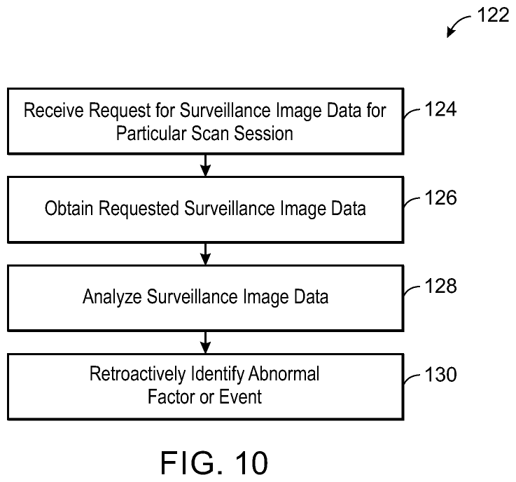

FIG. 10 is a flowchart of a method 122 for monitoring an environment within a scan room having a medical imaging scanner (e.g., retroactively). The method 122 may be performed by one or more components (e.g., processing circuitry) of the CT imaging system 10 in FIGS. 1 and 2 or another type of imaging system. One or more steps of the method 122 may be performed simultaneously and/or in a different order than depicted in FIG. 10. In certain embodiments, the method 122 may be performed by one or more components (e.g., processing circuitry) of a remote computing device.

The method 122 includes receiving a request for surveillance image data for a particular scan session (block 124). The particular scan session may be associated with a scan where something was not optimal prior to and/or during the scan, which may have resulted in sub-optimal image quality of image data acquired from the scan or improper performance of a component of the medical imaging scanner for an unknown reason. The surveillance image data from the scanning session is of a scan room from an optical imaging system disposed within the scan room where the imaging session occurred. The optical imaging system may include one or more cameras or sensors (e.g., video cameras, three-dimensional (3D) camera, LiDAR camera, etc.) located within the scan room. In certain embodiments, one or more cameras may be disposed on a component of the medical imaging scanner. For example, a camera may be located on a top of a gantry. In certain embodiments, one or more cameras may be located within the scan room at a location separate from the medical imaging scanner. For example, a camera may be located on a ceiling in the scan room. In certain embodiments, cameras may be disposed on a component of the medical imaging scanner and at a location separate from the medical image scanner. In certain embodiments, the surveillance image data is video data. In certain embodiments, the surveillance image data is individual frames or images.

The method 122 also includes obtaining the requested surveillance image data for the particular scan session (block 126). In certain embodiments, the requested surveillance image data may be retrieved from a memory or database of the medical imaging system that the scan occurred with. In certain embodiments, the requested surveillance image data may be retrieved from a memory or database located at location remote from the medical imaging system (e.g., remote from the facility having the medical imaging system).

The method 122 further includes analyzing the requested surveillance image data for any abnormal factor or event within the environment that potentially adversely impacted the scan of the subject (e.g., patient) with the medical imaging scanner (block 128). The method 122 even further includes retroactively identifying any abnormal factor or event within the environment of the scan room that potentially adversely impacts a scan of a subject (e.g., patient) with the medical imaging scanner based on the analysis of the requested surveillance image data (block 130). In certain embodiments, a probability may be calculated for a particular event in order to identify a factor or event as an abnormal factor or event that potentially adversely impacts the scan. In certain embodiments, comparing current surveillance image data of the scan room to reference image data (e.g., where environment conditions are considered normal) may be utilized in identifying a factor or event as an abnormal factor or event that potentially adversely impacts the scan. An abnormal factor or event potentially adversely impacts the scan by adversely impacting an image quality of an image data obtained during the scan, potentially adversely impacting a safety of the subject, and/or potentially adversely impacting setting up or conducting the scan.

In certain embodiments, the identified abnormal factor or event in the requested surveillance image data may have occurred in the presence or the absence of the subject to be imaged. In certain embodiments, the identified abnormal factor or event in the requested surveillance image data may have occurred prior to the scan. In certain embodiments, the identified abnormal factor or event in the requested surveillance image data may have occurred during the scan.

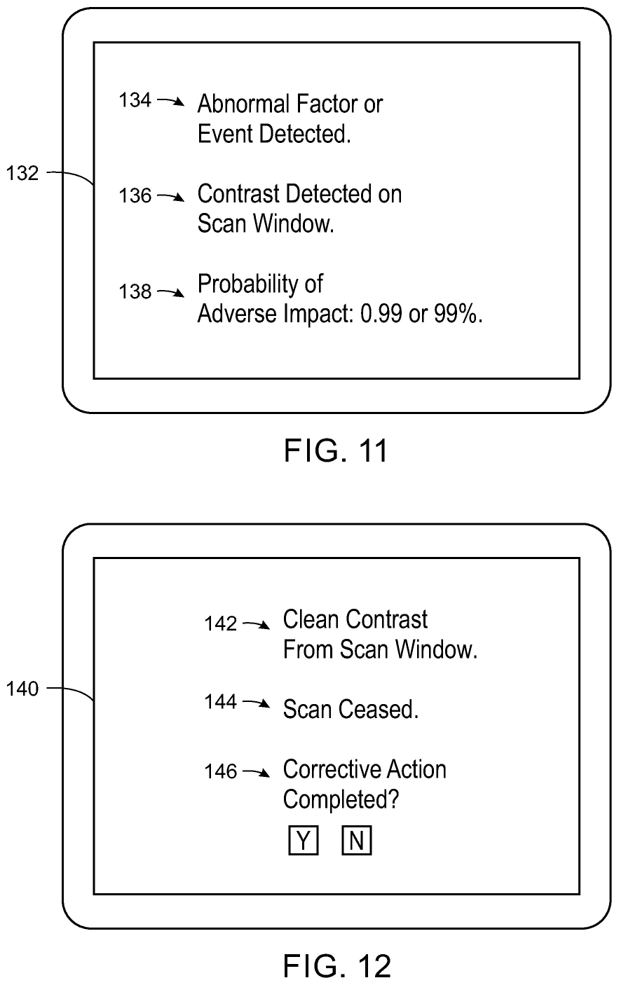

FIG. 11 is a schematic diagram of a user interface 132 providing a notification of an abnormal factor or event identified in an environment within a scan room having a medical imaging scanner that potentially adversely impacts a scan. The user interface 132 may be displayed on a display of a console of the operator workstation or a separate computing device (e.g., remote computing device). As depicted, the user interface 132 includes a notification 134 indicating that an abnormal factor or event has been identified in the environment of the scan room. As depicted, the notification 134 may also include a description 136 of the identified abnormal factor or event. The identified abnormal factor or event may be any number of different items. For example, one or more objects may be in the way when moving components (e.g., table or cradle) of the medical imaging scanner or located in an undesirable location (e.g., behind a gantry). For example, a boom, a chair, or straps may be in the way or impede the movement of a component of the medical imaging scanner (e.g., table or cradle). Another example of an abnormal factor or event may be a risk of pulling an object or device (e.g., ventilator tube, intravenous line, etc.) from the subject (e.g., patient) when moving the table. A further example of an abnormal event or factor would be an individual entering the scan room during a scan. An even further example of an abnormal event or factor may be contrast or bodily fluids located on the scan window or another component of the medical imaging scanner. A yet further example of an abnormal event or factor may be a dent in a component of the medical imaging scanner. In certain embodiments, a calculated probability 138 (e.g., probability score) may be provided that the abnormal event or factor adversely affects the scan of the subject (e.g., patient).

FIG. 12 is a schematic diagram of a user interface 140 providing a corrective action for an abnormal factor or event identified in an environment within a scan room having a medical imaging scanner that potentially adversely impacts a scan. The user interface 140 may be displayed on a display of a console of the operator workstation or a separate computing device (e.g., remote computing device). As depicted, the user interface 140 includes a corrective action 142 for rectifying an abnormal factor or event identified in the environment within the scan room having the medical imaging scanner. As an example, the operator may be instructed to clean contrast off the scan window. In another example, the operator may be instructed to move a chair. In certain embodiments, the user interface 140 includes a notice 144 that an action related to the scan was ceased before the identified abnormal factor or event adversely affects the scan. For example, the scan may be stopped if somebody enters the scan room. In another example, automatic movement of the table may be ceased and the operator may have to enter the scan room to manually move the table via the controls associated with the table. In certain embodiments, the user interface 140 may require the operator to provide an input (e.g., making or inputting a selection) that the corrective action was taken before the scan can proceed as indicated by reference numeral 146. In certain embodiment, one or more of the items shown in the user interfaces 132 and 140 in FIGS. 11 and 12, respectively may be shown together on the same user interface.

Technical effects of the disclosed embodiments include providing systems and methods for monitoring an environment within a scan room having a medical imaging scanner for any abnormal factor or event that may potentially adversely impact a scan of an object with the medical imaging scanner. In particular, the disclosed techniques include receiving surveillance image data of the scan room from an optical imaging system disposed within the scan room, identifying any abnormal factor or event within the environment that potentially adversely impacts a scan of an object with the medical imaging scanner, and recording (e.g., logging) any identified abnormal factor or event to an event log file. Technical effects of the disclosed embodiments may include proactively identifying an abnormal factor or event, thus, enabling the rectification of the abnormal factor or event to keep the scan from being adversely impacted. Technical effects of the disclosed embodiments may include retroactively identifying an abnormal factor or event that may have impacted a scan so that future similar abnormal factors or events may be proactively identified and rectified to keep a future scan from being adversely impacted.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform] ing [a function] . . . ” or “step for [perform] ing [a function] . . . ”, it is intended that such elements are to be interpreted under 35 U.S.C. 112 (f). However, for any claims containing elements designated in any other manner, it is intended that such elements are not to be interpreted under 35 U.S.C. 112 (f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for monitoring an environment within a scan room having a medical imaging scanner, comprising:
receiving, at a processor, surveillance image data of the scan room from an optical imaging system disposed within the scan room;
identifying, via the processor, any abnormal factor or event within the environment that potentially adversely impacts a scan of a subject with the medical imaging scanner based on the surveillance image data;
causing, via the processor, a notification of an identified abnormal factor or event to be provided to an operator of the medical imaging scanner; and
recording, via the processor, any identified abnormal factor or event to an event log file.

2. The computer-implemented method of claim 1, wherein identifying any abnormal factor or event within the environment occurs prior to the scan.

3. The computer-implemented method of claim 1, wherein the identifying any abnormal factor or event within the environment occurs prior to and during the scan.

4. The computer-implemented method of claim 1, wherein potentially adversely impacting the scan comprises potentially adversely impacting an image quality of image data obtained during the scan, potentially adversely impacting a safety of the subject, or potentially adversely impacting setting up the scan or conducting the scan.

5. The computer-implemented method of claim 1, further comprising causing, via the processor, a corrective action for the operator to perform to rectify the identified abnormal factor or event to be provided to the operator of the medical imaging scanner.

6. The computer-implemented method of claim 1, further comprising proactively ceasing, via the processor, an action related to the scan before the identified abnormal factor or event adversely affects the scan.

7. The computer-implemented method of claim 1, wherein the optical imaging system comprises one or more cameras disposed on a component of the medical imaging scanner and/or within the scan room at a location separate from the medical imaging scanner.

8. The computer-implemented method of claim 1, wherein the medical imaging scanner comprises a computed tomography scanner.

9. The computer-implemented method of claim 1, wherein identifying any abnormal factor or event within the environment that potentially adversely impacts the scan comprises calculating a probability of a particular factor or event adversely impacting the scan.

10. A system for monitoring for monitoring an environment within a scan room having a computed tomography scanner, comprising:
an optical imaging system disposed within the scan room;
a memory encoding processor-executable routines; and
a processor configured to access the memory and to execute the processor-executable routines, wherein the processor-executable routines, when executed by the processor, cause the processor to:
receive surveillance image data of the scan room from the optical imaging system;
identify any abnormal factor or event within the environment that potentially adversely impacts a scan of a subject with the computed tomography scanner based on the surveillance image data; and
record any identified abnormal factor or event to an event log file.

11. The system of claim 10, wherein the processor-executable routines, when executed by the processor, cause the processor to cause notification of an identified abnormal factor or event to be provided to an operator of the computed tomography scanner.

12. The system of claim 11, wherein the processor-executable routines, when executed by the processor, cause the processor to cause a corrective action for the operator to perform to rectify the identified abnormal factor or event to be provided to the operator of the computed tomography scanner.

13. The system of claim 11, wherein the processor-executable routines, when executed by the processor, cause the processor to proactively cease an action related to the scan before the identified abnormal factor or event adversely affects the scan.

14. The system of claim 10, wherein identifying any abnormal factor or event within the environment that potentially adversely impacts the scan occurs prior to and/or during the scan.

15. The system of claim 10, wherein identifying any abnormal factor or event within the environment that potentially adversely impacts the scan occurs subsequent to the scan.

16. The system of claim 10, wherein identifying any abnormal factor or event within the environment occurs prior to the scan.

17. The system of claim 10, wherein potentially adversely impacting the scan comprises potentially adversely impacting an image quality of image data obtained during the scan, potentially adversely impacting a safety of the subject or potentially adversely impacting setting up the scan or conducting the scan.

18. The system of claim 10, wherein the optical imaging system comprises one or more cameras disposed on a component of the computed tomography scanner and/or within the scan room at a location separate from the computed tomography scanner.

19. The system of claim 10, wherein identifying any abnormal factor or event within the environment that potentially adversely impacts the scan comprises calculating a probability of a particular factor or event adversely impacting the scan.

20. A non-transitory computer-readable medium, the computer-readable medium comprising processor-executable code that when executed by a processor, causes the processor to:
receive surveillance image data of a scan room having a medical imaging scanner from an optical imaging system disposed within the scan room;
identify any abnormal factor or event within an environment of the scan room that potentially adversely impacts a scan of a subject with the medical imaging scanner based on the surveillance image data;
cause a notification of an identified abnormal factor or event to be provided to an operator of the medical imaging scanner; and
record any identified abnormal factor or event to an event log file.
